(19) United States
(12) Patent Application Publication
(10) Pub. No.: US 2021/0338145 A1
(43) Pub. Date: Nov. 4, 2021

(54) Fetal Monitoring Device

(71) Applicant: Kimberly Dehaney, Roosevelt, NY (US)

(72) Inventor: Kimberly Dehaney, Roosevelt, NY (US)

(21) Appl. No.: 17/197,250

(22) Filed: Mar. 10, 2021

Related U.S. Application Data
(60) Provisional application No. 63/018,644, filed on May 1, 2020.

(51) Int. Cl. A61B 5/00; A61B 5/024; A61B 5/11

(52) U.S. Cl. CPC A61B 5/4362; A61B 5/0022; A61B 5/0011; A61B 2560/0214; A61B 5/11; A61B 5/6898; A61B 5/6831; A61B 5/02411

(57) ABSTRACT

This present invention discloses a fetal monitoring device that enables the monitoring of an unborn baby's heartbeat and its movements inside the womb. The fetal monitoring device includes an elastic strap with a fetal monitor disposed centrally on the strap. The fetal monitoring device is worn such that the strap fits to the abdomen of the pregnant woman, and the fetal monitor is placed adjacent to the location of the fetus, so as to monitor the fetus' condition and further project real-time imagery of the unborn baby on a related display device.

### CROSS-REFERENCE TO RELATED APPLICATION

The present application claims priority to, and the benefit of, U.S. Provisional Application No. 63/018,644, which was filed on May 1, 2020 and is incorporated herein by reference in its entirety.

### BACKGROUND

The present invention relates generally to the field of fetal monitoring. More specifically, the present invention relates to a device for monitoring fetal conditions, e.g. heartbeats, contraction patterns or real-time visual monitoring of a fetus during pregnancy and child birth at home. The device is a conformable belt device worn around the stomach or abdomen of a pregnant woman such that a monitoring unit of the device is placed adjacent to the fetus to monitor its condition. Accordingly, the present specification makes specific reference to the device described herein and it is to be appreciated that aspects of the present invention are also equally amenable to other similar applications, devices and methods of use.

Fetal movement, uterine contraction, and fetal heart rate are three of the major physiological parameters to understand a fetus' condition during a pregnancy. More specifically, fetal movement refers to a fetus' movement in the uterus, uterine contraction refers particularly to the pressure generated by such contraction, and fetal heart rate refers to the speed or pace of the fetus' heartbeats. Of the three parameters, fetal movement provides the earliest and most readily detectable signal, and hence constitutes the most clinically recommended method for a pregnant woman or other individual to monitor the unborn baby's health. This explains why pregnancy instruction manuals typically include a fetal movement record and encourage an expectant mother and other concerned individuals to measure fetal movement regularly to ensure the fetus' safety. However, continually measuring and recording fetal movement by oneself can be very time-consuming. For example, during the eighth week of pregnancy, there is one fetal movement at least every 13 minutes. In the twentieth week, the average number of fetal movements per 12 hours is about 200, and the number increases to about 575 in the thirty-second week of the pregnancy. For a pregnant woman, therefore, counting the number of fetal movements is by no means convenient or accurate. Not only is the identification of fetal movement highly subjective, but also the prolonged counting process is difficult to carry out on a daily basis and the individual may forget to record sufficient information to assist in the care of the unborn child.

By way of background, pregnant women routinely go for continual or periodic checkups which include fetal monitoring sessions, as well as checking the general health of the mother, or patient in the case of a surrogate, during pregnancy. Fetal monitoring is also performed on the patient during some prenatal consultations, and enables a practitioner to assess the condition such as to continuously record the heartbeat of the fetus and the contraction patterns of the woman's uterus during antepartum, labor and delivery, and advise the pregnant woman accordingly. A monitoring session is a way to check on the activity and/or health of the fetus and shows if the fetus is doing well or is having any complications. The monitoring session also allows the pregnant woman to interact with her unborn baby in some way, and also reassures the mother that the fetus is active and doing well and that the pregnancy is proceeding normally.

Accordingly, pregnant women should visit either a physician's office or a designated fetal monitoring center for regular checkups and/or fetal monitoring. A pregnant woman usually has to make an appointment before visiting her doctor for a monitoring session, and such sessions are often costly and require a minimum of 45 minutes, and up to 4 hours, for each monitoring session. If the patient needs to move during the monitoring session, the complete setup likely needs to be removed and placed back again, which adds inconvenience, additional time and cost burden to both the medical facility and the woman being monitored. Further, if the fetal monitoring center is far away from the expectant mother's residence, then the entire process ends up taking more than half of the day due to the increased travel requirements. Also, such centers or hospitals are often difficult for patients to access (e.g., during the ongoing COVID-19 pandemic), and typically require someone to accompany the pregnant woman to the medical facility to have the fetus checked. The expensive, inconvenient and time-consuming nature of the current fetal monitoring sessions places a great burden on the pregnant woman and any parties involved. Currently, it is not possible to setup a remote fetal monitoring station in the home, and pregnant women cannot otherwise experience the connection to the fetus in the womb without significant inconvenience, time and expense.

With traditional systems, monitoring of pregnant mothers who are not categorized as high risk is typically limited to a few regularly scheduled times during the course of the pregnancy. This, in turn, leads to potentially reduced efficacy of monitoring in terms of missing potential signs of distress or other indicators about the well-being of the unborn child. Further, immobility of the traditional system leads to limited access to the healthcare system for pregnant mothers in remote areas and/or in underserved areas. In fact, sometimes, such pregnant women are not tested or monitored at all, which may cause later issues or problems for the mother and/or the baby.

Typically, ultrasonic and electronic fetal heart rate monitoring are used in healthcare centers to assess fetal well-being prior to and during labor. Machines and equipment for fetal monitoring are relatively bulky and expensive, and the process for monitoring the health of the fetus is quite complicated and can be done usually only under the supervision of a qualified health professional or a doctor. The pregnant woman can only monitor and access real-time visual representation or on-screen movements of the baby during these periodic routine checkup sessions in healthcare centers and cannot observe the baby and monitor the unborn baby's movements on a regular daily basis at home.

Conventionally, Doppler ultrasound is used to extract information regarding fetal health. However, such technologies and related equipment require bedside monitoring which is normally limited to hospital settings. Further, the traditional fetal monitoring setup is time-consuming and does not allow immediate attachment of the monitoring equipment in case of any emergency or complication in a pregnant woman's condition. A monitoring device which can be quickly set up is preferred for the pregnant woman, and can effectively check the unborn baby's condition in case of emergency.

Therefore, there exists a long felt need in the art for a convenient and effective fetal monitoring device that allows a pregnant woman to check the condition of her fetus at home. There also exists a long felt need in the art for a fetal monitoring device that is easily accessible, and minimizes visits to healthcare centers, which in turn reduces expense to the expectant mother. Furthermore, there exists a long felt need in the art for a fetal monitoring device which is lightweight and that can be easily set up by an expectant mother at home, and that does not require a healthcare professional to set up the device or equipment for a fetal monitoring session. Moreover, there is a long felt need in the art for a fetal monitoring device that reduces the cost of monitoring sessions, doctor's appointments, and other costly charges. Additionally, there is a long felt need in the art for a fetal monitoring device which can be quickly set up in case of emergency or exigent situations when the pregnant woman is at home and needs immediate information to assist the healthcare professionals in the treatment of the unborn child. Finally, there is a long felt need in art for a fetal monitoring device which effectively monitors various fetal conditions, such as the unborn baby's heartbeat, contraction patterns and/or real-time visual monitoring of the fetus during pregnancy and child birth at home.

The subject matter disclosed and claimed herein, in one embodiment thereof, comprises a fetal monitoring device that enables pregnant women to monitor their unborn baby's condition, and enables the expectant mother to visually monitor her unborn baby's movements in real-time situations and at home. The device of the present invention allows expectant mothers and family members to monitor and develop a bond with their unborn baby in a personal, intimate, and exciting way. The fetal monitoring device includes a monitoring device/unit that is attached or otherwise connected to an elastic strap. The elastic strap is attached comfortably to the pregnant woman's stomach or abdomen such that the monitoring device is placed immediately adjacent to the location of the fetus to monitor its ongoing condition. The monitoring unit further comprises a fetal monitoring transducer and other sensors to monitor the unborn baby's heartbeat and other vital signs in real time, and includes a wired or wireless connectivity mechanism to connect the fetal monitoring device to a display in order to show the unborn baby's movements on the device's display.

In this manner, the fetal monitoring device of the present invention accomplishes all of the forgoing objectives, and provides a relatively quick and easy solution to monitor an unborn baby's condition, heartbeats and view the unborn baby's movements in real-time according to the preferences of the mother or other caregiver. The portable fetal monitoring device offers peace of mind to the expectant mother and family members that the unborn baby is healthy and active inside the womb, and delivers a sense of calm from the comfort of their own home. The device can be easily set up at home and does not require any healthcare professional supervision for fetal monitoring. The fetal monitoring device is cost-effective, portable and lightweight, and is accessible to pregnant women in remote and/or underserved areas.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one embodiment thereof, comprises a fetal monitoring device that enables pregnant women to monitor their unborn baby's condition, heart beat and that enables a mother to visually monitor her baby's movements in real-time and at home. The device allows expectant mothers and family members to enhance the bond with their unborn baby in a personal, intimate, and exciting way. The fetal monitoring device has a monitoring device/unit attached to an elastic strap. The elastic strap is attached comfortably and easily to the pregnant woman's stomach such that the monitoring device is placed adjacent to the location of the fetus to monitor its ongoing condition. The monitoring unit further includes a fetal monitoring transducer and other sensors (e.g., an ultrasound piezoelectric transducer) to monitor the unborn baby's heartbeat in real time, and includes a wired or wireless connectivity mechanism to connect the fetal monitoring device to a display in order to show the baby's activities on the device's display.

In one exemplary embodiment, a fetal monitoring device is provided and includes a monitoring unit and an elongated strapping means. The monitoring unit includes sensors such as a fetal monitoring transducer, etc. to monitor the unborn baby's condition. The strapping means enables an expectant mother to comfortably, easily and securely wear the fetal monitoring device over her stomach or abdomen. The strapping means is an elastic strap having two ends, with an adjustable fastening mechanism to securely hold the device in the proper position. The monitoring device/unit is a circular shaped body, and includes various components for health monitoring, wired/wireless connectivity, device powering and charging, a real-time display mechanism and other features which may be desirable to have for the device. The device is disposed centrally on the elongated strapping means and may be adjusted as required to find the most suitable location to optimize the monitoring of the unborn child.

In a still further exemplary embodiment of the present invention, a fetal monitoring device, is provided and includes a detachable fetal monitoring unit and an elastic strap/belt. The elastic strap has a bracket for accommodating the detachable monitor unit inside the bracket structure. The bracket structure is positioned centrally on the elastic strap and allows the user of the device to attach or detach the fetal monitor unit to the strap. Further, the bracket structure position can be adjusted for the comfort and requirements of the user. The elastic strap further includes different mechanisms such as hook-and-loop fasteners, buttonholes, clasps and other suitable fastening means known in the art for adjusting the length of the strap to fit the stomach or abdomen of the pregnant woman in various stages of the pregnancy, thereby allowing the device to be fitted at various times during the pregnancy as the abdomen grows. The strap of the detachable fetal monitor unit is also washable.

In a yet another exemplary embodiment of the presently described invention, a fetal monitoring device is provided and includes a method of monitoring one or more fetal conditions including, for example and without limitation, the heartbeat of the unborn baby, the pregnant woman's own condition, and other important conditions and vital signs. The fetal monitoring device includes an elastic strap that is worn around the pregnant woman's stomach or abdomen, and is adjusted to comfortably fit the pregnant woman as the unborn child continues to grow during the pregnancy. Next, the strap with a fetal monitor unit is moved to the correct position so that the monitor unit is positioned over the desired location of the fetus. Finally, the fetal monitor unit is placed over the location of the fetus to monitor the unborn baby's health, condition, the pregnant woman's health and condition, and to display the unborn baby's movement and health condition on a display unit in real-time.

In yet another exemplary embodiment of the presently described invention, a fetal monitoring device is provided and includes a method of monitoring at least one of several conditions, including the heartbeat of the unborn baby, the pregnant woman's condition and other predetermined factors and conditions. The fetal monitoring device has an elongated elastic strap/belt with a centrally disposed bracket for accommodating a fetal monitor unit which is then attached to the stomach or abdomen of a pregnant woman. The fetal monitor unit is placed in the bracket and the strap with the monitor unit being worn around the pregnant woman's stomach or abdomen such that the fetal monitor unit is adjacent to the location of the fetus' position. Next, the fetal monitor unit is turned on, and is connected (hardwired or wirelessly) to a display device. Finally, the fetal and/or other selected conditions are monitored and are displayed on the connected display device.

In an exemplary embodiment of the presently described invention, a system for remote monitoring of a pregnancy, is presented and includes a fetal monitor that has at least one sensor. The fetal monitor further includes a wired or wireless connected module and at least one other module. The fetal monitor is removably attached to an expectant mother by a flexible strap and the monitor is held to the strap by a bracket. A display is the connected (via wires or wirelessly) to the fetal monitor and a data processing platform, remote from the fetal monitor. The data processing platform receives data from the at least one sensor, and from the at least one other module. A processor for processing the data and a controller for controlling and monitoring the fetal monitor is also provided. The at least one sensor and the connectivity module, and the at least one other module are all controlled by the controller, and the display displays the data collected by the fetal monitor.

In a further exemplary embodiment of the presently described invention, a method of remote monitoring a pregnancy is described, and includes the initial step of providing a fetal monitor that has at least one sensor. The fetal monitor further includes a wired or wireless connectivity module, and at least one other module. The fetal monitor is removably attached to an expectant mother by a flexible strap, and the fetal monitor is held to the strap by a bracket. The method further includes connecting the display wirelessly to the fetal monitor, and then providing a data processing platform, which is remote from the fetal monitor and the display. The data processing platform receives data from the at least one sensor and from the at least one other module. The data received from the fetal monitor is then processed while the controller controls the fetal monitor and the at least one sensor, and the at least one of the connectivity module and the at least one other module to provide data for display. Finally, the collected data is then displayed on a remote display for use by the expectant mother and her healthcare professionals.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one possible embodiment of a fetal monitoring device of the present invention in accordance with the disclosed structure;

FIG. 2 illustrates a perspective view of one possible embodiment of a fetal monitor of the fetal monitoring device of the present invention in accordance with the disclosed structure;

FIG. 3 illustrates a perspective view of another possible embodiment of a fetal monitoring device of the present invention in accordance with the disclosed structure;

FIG. 4 illustrates a perspective view of a pregnant woman wearing the fetal monitoring device of the present invention in accordance with the disclosed structure;

FIG. 5 illustrates a perspective view of a pregnant woman wearing the fetal monitoring device of the present invention and the display portion displaying the unborn baby's movements in accordance with the disclosed structure; and

FIG. 6 illustrates a perspective view of one potential embodiment of the display portion of the fetal monitoring device of the present invention in accordance with the disclosed structure and displaying a vital sign of the unborn baby being monitored.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof.

As noted above, there exists a long felt need in the art for an easy to use fetal monitoring device that allows a pregnant woman to check certain conditions of her unborn child at home. There also exists a long felt need in the art for a fetal monitoring device that is easily accessible, and that minimizes the inconvenience caused by frequent visits to a healthcare center for fetal monitoring. Furthermore, there exists a long felt need in the art for a fetal monitoring device which is lightweight and that can be easily set up by a user at home, and that does not require a healthcare professional to set up the device or equipment for a fetal monitoring session. Additionally, there is a long felt need in the art for a fetal monitoring device that reduces the expense of monitoring sessions, doctor's appointment, and other related costs and charges typically associated with obtaining vital information about the pregnancy. Moreover, there is a long felt need in the art for a fetal monitoring device which can be quickly and easily set up in case of emergency or when any other complications may occur when the pregnant woman is at home. Finally, there is a long felt need in art for a fetal monitoring device which effectively monitors different fetal conditions, the unborn baby's heartbeat, contraction patterns and/or real-time visual monitoring of the unborn baby during pregnancy and child birth at home.

The innovative product of the present invention features a fetal monitoring device that enables pregnant women to monitor their unborn baby's condition and vital signs, and that enables the mother or other concerned individual to visually monitor the unborn baby's movements in real-time and from home. The monitoring device allows expectant mothers and family members to bond with their unborn baby in a personal, intimate, and exciting way. The fetal monitoring device comprises a monitoring device/unit attached to an elastic strap. The elastic strap is attached comfortably to the pregnant woman's stomach or abdomen, such that the monitoring device is placed adjacent to the location of the fetus to monitor one or more fetal conditions and/or vital signs. The monitoring unit further comprises one or more fetal monitoring transducers and other sensors to monitor the unborn baby's vital signs in real time, and includes a wired or wireless connectivity mechanism to connect the fetal monitoring device to a display in order to show the unborn baby's movements and other activities on the display device. The elastic strap or the adjustable size strap enables the device to be attached to the abdomen or stomach of a pregnant woman during various stages of the pregnancy as the abdomen continues to grow in size as the unborn baby's development progresses. The monitoring device is portable, and can be easily set up at home without requiring the supervision of a healthcare professional.

The present invention, in one exemplary embodiment, is a portable fetal monitoring device that includes an adjustable elastic strap or belt with an integrated or detachable fetal monitor unit. The fetal monitoring device is worn by a pregnant woman in a manner such that the strap surrounds the stomach or abdomen of the pregnant woman, and the fetal monitor unit is positioned immediately adjacent to the location of the fetus. The fetal monitoring unit and its components enables easy monitoring of fetal conditions, unborn baby's heartbeats, fetal health, and the like, and enables a user to view the unborn baby activities and movements on a display device that is in electrical communication (wired or wireless) with the monitoring unit. The fetal monitoring device enables the pregnant woman, family members and medical professionals to hear the unborn baby's heartbeats on the go, and to monitor other conditions at the convenience of the user.

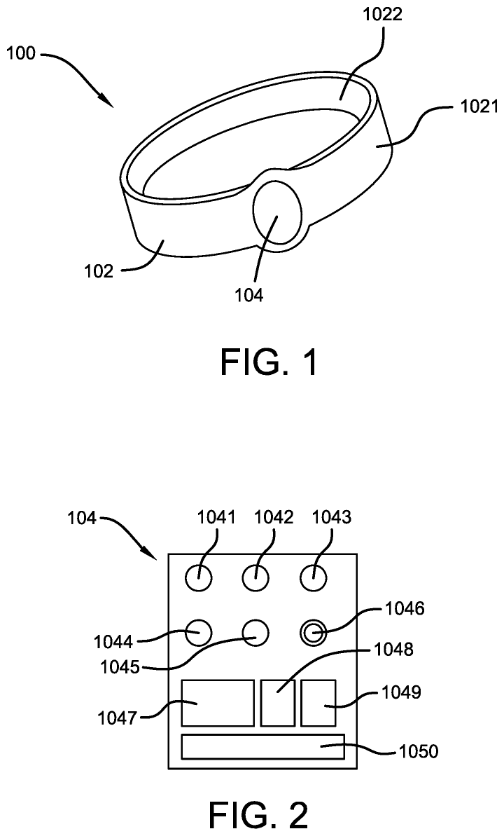

Referring initially to the drawings, FIG. 1 illustrates a perspective view of one possible embodiment of a fetal monitoring device of the present invention in accordance with the disclosed structure. The fetal monitoring device 100 includes an elongated elastic strap or belt 102 with a fetal monitor 104 integrated centrally to monitor fetal conditions. The fetal monitoring device 100 is placed on the stomach or abdomen of a pregnant woman such that the strap 102 surrounds the waist area of the pregnant woman, and the fetal monitor 104 is positioned adjacent to the location of the fetus of the pregnant woman to effectively monitor the expectant mother and the fetus' health, vital signs and other conditions. The strap 102 includes an outer surface 1021, which can have any design, prints, customized or personalized color, or user desired information. Further, an inner surface 1022 of the strap 102 is preferably soft and comfortable, and touches the skin of the stomach of the pregnant woman and can be covered with a soft fabric, such as terrycloth, cotton, woven or nonwoven material, non-allergenic material or other material which is suitable for a particular expectant mother.

Notwithstanding, the strap 102 is preferably made of an elastic material, and can be without limitation, made of materials such as plastic, polyurethane, polypropylene, polyethylene, silicone, elastic fabric, foam material and/or a foam-like material, or the like. The elastic strap 102 easily and comfortably fits around the stomach/abdomen area of the expectant mother's waist during the various stages of the pregnancy and as the unborn child continues to grow. The strap 102 may of any color, shape, pattern, and/or size to satisfy user need and/or preference.

In a preferred embodiment, the length of strap 102 may be 120 cm or less, such as 100 cm or less, but may also be any other suitable length. The width of the strap 102 is preferably 2-9 cm, and more preferably 4-7 cm, but may also be any other suitable width. The thickness of the strap 102 is preferably 1-8 mm, and more preferably 3-6 mm, but may also be any other suitable thickness. The above referenced dimensions have proven to be very comfortable for most expectant mothers, though other dimensions are also contemplated to fit other expectant mothers throughout a pregnancy. Furthermore, fetal monitoring devices 100 with these dimensions will have sufficient holding power for safe and secure attachment of the fetal monitor 104 to the body of the expectant mother and to retain the fetal monitor 104 in the proper position relative to the fetus during the data capture period.

The fetal monitor 104 itself is preferably a generally circular or round shaped structure comprising various sensors, a sound module, a device powering unit, one or more charging modules, a battery, and other components that enables a user to monitor different conditions of the fetus, a pregnancy or child birth labor at a remote location (e.g., the expectant mother's home). Notwithstanding, the shape of the fetal monitor 104 of the fetal monitoring device 100 may be other geometric shapes including, without limitation, an oval, a rectangular, a square, a triangle, etc., or a non-geometric shape, provided that the size and shape of the same is capable of monitoring the desired parameters. Additionally, the fetal monitor 104 may have a plastic or metallic body with the various components held inside or otherwise secured to the body of the fetal monitor 104. The fetal monitor 104 is the core of the fetal monitoring device 100, is relatively lightweight and allows a user to, among other things, hear an unborn baby's heartbeat and view the unborn baby's activities and movements on a display device in communication with the fetal monitor 104 of the fetal monitoring device 100, as explained more fully below.

FIG. 2 illustrates a perspective view of a fetal monitor 104 of the fetal monitoring device 100 of the present invention in accordance with the disclosed structure. As referenced above, the fetal monitor 104 includes, for example, an electrocardiogram sensor 1041, a motion sensor 1042, an ultrasound sensor 1043, a sound module 1044, a temperature sensor 1045, a power switch 1046, a processor 1047, a controller 1048, a wireless connectivity module 1049, a battery 1050 and other components to enable effective fetal monitoring of an unborn child. The fetal monitor 104 is compact and lightweight and can be easily activated using the power switch 1046 by pressing an external power button (not shown) on the fetal monitor 104.

The electrocardiogram (ECG) sensor 1041 monitors the heartbeat of the unborn baby, wherein the motion sensor 1042 tracks unborn baby kicks, contractions and other movements inside the womb of the pregnant woman, and the ultrasound sensor 1043 both sends and receives ultrasonic waves into the pregnant woman's womb for producing visual images of the fetus and the surrounding organs. These real time images showing the appearance and movement of the fetus allow for observation of the development of the fetus. The sound module 1044 comprises acoustic sensors that, for example, record acoustic signals emitted by the unborn baby's heart, and is primarily utilized for monitoring the unborn baby's heartbeat. The sound module 1044 further comprises speakers to play the monitored heartbeat of the unborn baby. The temperature sensor 1045 monitors, among other things, the temperature of the pregnant woman's body.

The data collected by the ECG sensor 1041, the motion sensor 1042, the ultrasound sensor 1043, the sound module 1044, the temperature sensor 1045, the power switch 1046 and wireless connectivity module 1049 and the other sensors that may also be included (not shown) are transmitted to the processor 1047 for further data processing and refinement. More specifically, the collected data or signals are processed at the processor 1047, which further sends a signal to the controller 1048 for activating different features of the fetal monitoring device 100. The fetal monitor 104 may have a cable connecting component to connect the fetal monitoring device to display devices such as monitor, a television, a mobile device, a tablet or the like, and may alternatively have a wireless connectivity module 1049 for connecting the fetal monitoring device 100 wirelessly. The various display devices may connect to the fetal monitoring device 100 through a suitable wireless communication technology such as Bluetooth, RFID, Wi-Fi, NFC or any other technology known in the art. Additionally, the fetal monitor 104 has a battery 1050 to power the device 100. The battery 1050 may be a NiMH battery, or any other type of batteries known in the art. However, a NiMH battery has properties that reduce the probability of overheating and, therefore, decrease recharging requirements versus using lithium-ion or lithium polymer batteries (LiPo). Nonetheless, a non-rechargeable or single use battery may also be used to power fetal monitoring device 100.

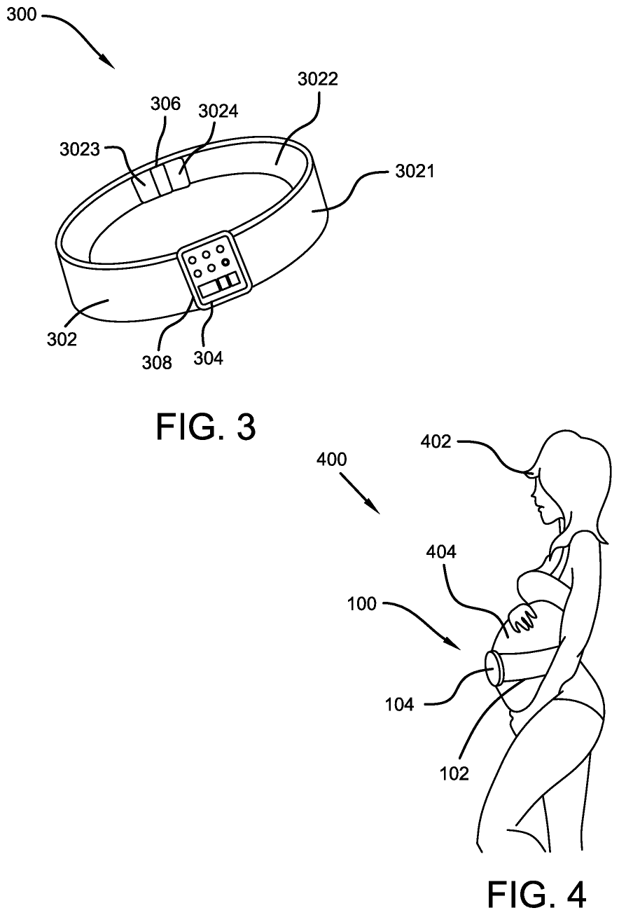

FIG. 3 illustrates a perspective view of another possible embodiment of a fetal monitoring device of the present invention in accordance with the disclosed structure. The fetal monitoring device 300 includes an elongated strap or belt 302 having a first end 3023 and a second end 3024. The strap or belt 302 includes an outer surface 3021 with any design, color, patterns, customized prints, etc. desired by a user, and an inner surface 3022 which is soft and gentle and comfortably attaches to the skin of the stomach or abdomen of the expectant mother. The strap 302 is preferably made of an elastic material and can be, without limitation, made of materials such as plastic, polyurethane, polypropylene, polyethylene, silicone, elastic fabric, foam material and/or a foam-like material, natural fibers, synthetic fibers, and combinations thereof or the like. The strap or belt 302 easily and comfortably fits around the waist of the expectant mother in the vicinity of her stomach and/or abdomen, and can be adjusted over time to address the varying stages of pregnancy as the unborn child continues to develop in vivo.

The strapping means 302 further includes a bracket 308, preferably disposed centrally along the strap 302 for accommodating a fetal monitor 304 easily. The bracket 308 may be made comprised of any material, such as hard plastic, metal or any other similar materials, that is capable of securing, and not interfering with the operation of, the fetal monitor 304. The bracket 308 is preferably of a shape generally corresponding to that of the fetal monitor 304, and is slightly larger in size so as to accommodate the fetal monitor 304. In this embodiment, the fetal monitor 304 is preferably a generally rectangular or square shaped structure with an outer body made of plastic or metal, including various sensor modules and components inside the body of the device. The fetal monitor 304 may be easily attached or removed from the bracket 308 during use of the fetal monitoring device 300 by the individual, and its construction should not interfere with the function or the fetal monitor 304 or its various components. The detachable fetal monitor 304 allows the user to wash and clean the strap 302 when the fetal monitor 304 is detached from the bracket 308, thereby ensuring that the fetal monitor 304 is not damaged. The strap or belt 302 further comprises a fastening mechanism 306, such as belt hooks or by a variety of other fasteners, including hook and loop fasteners (e.g. Velcro® material), buckle mechanisms, snaps, buttons, hooks or the like, to attach the two ends 3023, 3024 of the strap 302.

As stated above, the fetal monitor 304 may comprise various sensors, a sound module, a device powering unit, charging modules, a battery, and other components that enable a user to monitor the condition of the expectant mother and the fetus during pregnancy and/or child birth. As also stated above, the fetal monitor 304 of this particular embodiment is the core of the fetal monitoring device 300, is relatively lightweight, and allows a user to hear the unborn baby's heartbeat and view the unborn baby's activities and movements on a display device attached to the fetal monitoring device (i.e., hardwired or wirelessly). Notwithstanding, the shape of the fetal monitor 304 of the fetal monitoring device 300 may be any shape such as oval, circular, square, rectangular, etc., and may also include other shaped configurations such as geometric structures or ergonomically sensitive structures to add to the comfort of the expectant mother.

FIG. 4 illustrates a perspective view of a pregnant woman 402 wearing the fetal monitoring device of the present invention in accordance with the disclosed structure. More specifically, the pregnant woman 402 wears the fetal monitoring device 100 having a strap 102 and a fetal monitor 104 removably attached thereto. As discussed above, the strap 102 is preferably elastic and comfortably fits around the waist of the woman 402 regardless of the size of the woman 402 during the varying stages of her pregnancy. The inner surface (not shown) of the strap 102 is soft and comfortably touches the skin of the stomach 404 of the wearer 402. As also discussed above, the fetal monitoring device 100 is worn around the stomach or abdomen 404 of the woman 402 to monitor the condition of the woman 402 and the fetus during the pregnancy and/or child birth process and while at a remote location, such as the woman's home. The fetal monitor 104 also allows the wearer 402 to hear the unborn baby's heartbeat, and view the unborn baby's activities and movements on a display device attached to the fetal monitoring device, preferably wirelessly.

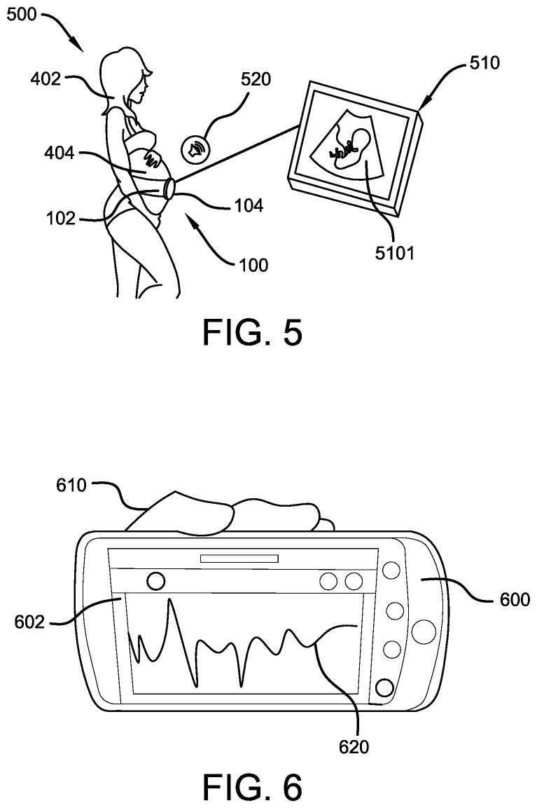

FIG. 5 illustrates a perspective view of a pregnant woman wearing the fetal monitoring device of the present invention and the display portion displaying the unborn baby's movements in accordance with the disclosed structure. The setup 500 shows a display device 510 wirelessly connected to the fetal monitoring device 100 and having a fetal monitor 104. The fetal monitor 104 includes various sensors to monitor the fetus health and conditions, and sends a signal to the controller (not shown) to display a real-time image and projection 5101 of the unborn baby in the womb of the pregnant woman 402. The display device 510 can be any device, such as a mobile device, a television, a monitor or the like, which is capable of displaying the fetus' condition in real-time. The display device 510 can be connected to the fetal monitor 104 by any wired or wireless means. As best shown in FIG. 5, the fetal monitor 104 includes acoustic and heartbeat sensors which measures and monitors the unborn baby's heartbeats, and enables the speakers in the fetal monitor 104 to amplify the heartbeat sound 520 which can be easily heard by the pregnant woman 402 and the family members or individuals at the remote site, such as the woman's home. Therefore, the fetal monitoring device 100 of the present invention accomplishes all of the forgoing objectives, and provides a relatively quick and easy solution to monitor the unborn baby's condition, and view the unborn baby's movements in real-time and according to the needs and preferences of the user. Finally, the portable fetal monitoring device 100 offers comfort in the form of peace of mind to the expecting mother 402 and family members that the unborn baby is healthy and active inside the mother's womb, from the convenience of their own home.

FIG. 6 illustrates a perspective view of one potential embodiment of the display portion 600 of the fetal monitoring device 100 of the present invention in accordance with the disclosed structure and displaying a vital sign 620 of the unborn baby being monitored. More specifically, the display device 600 can be any device, such as a mobile device, a television, a monitor or the like, which is capable of displaying the fetus' condition in real-time. The display device 600 can be connected to the fetal monitor 104 by any wired or wireless means, as described above. For example, the display device 600 may be a smart phone that has a screen 602 that displays the fetal heartbeat 620 in the form of a known ECG waveform. The device 600 may comprise an interactive touch screen display, and may further include encoding functionality to permit the secure transmission of fetal heartbeat 620 from the fetal monitor 104 to the device 600.

In an embodiment of the present invention, the functions and settings of the fetal monitor 104 can be changed or set by a corresponding smartphone mobile application installed in a mobile device of the user. The smartphone may connect to the fetal monitor 104 of the fetal monitoring device 100 through a suitable wireless communication technology such as Bluetooth, RFID, Wi-Fi, NFC, Wi-Fi Direct, a cellular network or any other suitable technology known in the art. The smart device/phone app may turn on the device 100, project real-time visualization of unborn baby, hear the unborn baby's heartbeats, or any other suitable setting required by the user. In a preferred embodiment, the corresponding smartphone mobile application logs the fetus' and pregnant woman's health data, and may also store the collected data in the application or send the data to a health dashboard on a remote server, such as that of a healthcare provider. The data stored on the servers may be utilized by health professionals to advise the pregnant woman during her pregnancy, as well as to determine if additional treatment or an office visit is necessary. As stated above, in this particular embodiment, the mobile device 600 is implemented as a smartphone, but is not limited thereto. For example, the mobile device may alternatively be a tablet, a computer, a personal digital assistant, a smart watch, etc. The mobile device may be pre-installed with a fetal movement algorithm for analyzing, making judgements about, and computing with the dynamic physiological signals of the pregnant women's abdomen/stomach received respectively by the fetal sensors in order to obtain accurate fetal movement information. For example, each fetal sensor may transmit the dynamic physiological signal sensed to the mobile device via the corresponding wireless connection module.

The mobile device may also operatively communicate with a storage platform, preferably cloud data storage, and may include File Transfer Protocol (FTP) server cloud data storage. Cloud computing in all of its forms may also be used for achieving the functionality of the systems and methods described herein.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “strap”, and “belt” are interchangeable and refer to the strap 102 of the present invention. As used herein “fetus monitor” refers a device comprising various sensor and components for enabling the objectives of the invention.

Notwithstanding the forgoing, the fetal monitoring device 100 of the present invention can be of any suitable size and configuration as is known in the art without affecting the overall concept of the invention, provided that it accomplishes the above stated objectives. One of ordinary skill in the art will appreciate that the size, configuration and material of the fetal monitoring device 100 as shown in the FIGS. are for illustrative purposes only, and that many other sizes of the fetal monitoring device 100 are well within the scope of the present disclosure. Although the dimensions of the fetal monitoring device 100 are important design parameters for user convenience, the fetal monitoring device 100 may be of any size that ensures optimal performance during use and/or that suits user need and/or preference.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A fetal monitor device for remote monitoring of a fetus, the fetal monitoring device comprising;
a flexible strap having a fastener and a bracket;
a device for monitoring one or more conditions of a fetus, the device is sized and configured so as to be retained in the bracket on the strap;
the device having a plurality of sensors with each sensor selected to monitor a specific condition of the fetus;
a power supply for supplying power to the device; and
a transmission module for transmitting data collected by the device to a display.

2. The fetal monitor device of claim 1, wherein the plurality of sensors include at least two of an electrocardiogram sensor, a motion sensor, an ultrasound sensor, and a temperature sensor.

3. The fetal monitor device of claim 1, wherein the device further comprises a sound module, a power switch, a processor and a controller.

4. The fetal monitor device of claim 1, wherein the power supply is one of a rechargeable or replaceable battery.

5. The fetal monitor device of claim 3, wherein the data collected is transmitted to the processor for data processing and the controller activates one or more of the plurality of sensors of the device based on the data.

6. The fetal monitor device of claim 1, wherein the device further comprises a display.

7. The fetal monitor device of claim 6, wherein the display is connected wirelessly to the device.

8. The fetal monitor device of claim 7, wherein the wireless connection is via one of a cellular network connection, a Bluetooth connection, a wi-fi connection, or a combination thereof.

9. A system for remote monitoring of a pregnancy comprising;
a fetal monitor having at least one sensor, a wireless connectivity module and at least one other module, wherein the fetal monitor is connected to an individual by a flexible strap and held to the strap by a bracket;
a display wirelessly connected to the fetal monitor;
a data processing platform, remote from the fetal monitor, wherein the data processing platform receives data from the at least one sensor and at least one other module;
a processor for processing the data;
a controller for controlling the fetal monitor and the at least one sensor and at least one of the connectivity module and the at least one other module; and
the display providing a display of the data collected by the fetal monitor.

10. The system for remote monitoring of a pregnancy of claim 9, wherein the fetal monitor is comprised of an electrocardiogram sensor, a motion sensor, an ultrasound sensor, and a temperature sensor.

11. The system for remote monitoring of a pregnancy of claim 9, wherein the fetal monitor further comprises at least one of a sound module, a power switch, a power supply, a processor and a controller.

12. The system for remote monitoring of a pregnancy of claim 9, wherein the wireless connection of the display is via one of a cellular network connection, a Bluetooth connection, a wi-fi connection or a combination thereof.

13. The system for remote monitoring of a pregnancy of claim 9, wherein the data processing platform is a cloud-based platform.

14. The system for remote monitoring of a pregnancy of claim 9, wherein the display is provided through at least one of a smart phone, a tablet, a smart watch, or a personal data assistant.

15. A method of remote monitoring a pregnancy comprising the steps of:
providing a fetal monitor having at least one sensor, a wireless connectivity module and at least one other module, wherein the fetal monitor is attached to an individual by a flexible strap and the fetal monitor is held to the flexible strap by a bracket;
connecting a display wirelessly to the fetal monitor;
providing a data processing platform, remote from the fetal monitor and the display, wherein the data processing platform receives data from the at least one sensor and at least one other module;
processing the data received from the fetal monitor;
controlling the fetal monitor by a controller, the controller controlling the at least one sensor and at least one of the connectivity module and the at least one other module to provide data for display; and
displaying the data collected by the fetal monitor on the display.

16. The method of claim 15, wherein the fetal monitor is comprised of an electrocardiogram sensor, a motion sensor, an ultrasound sensor, and a temperature sensor.

17. The method of claim 15, wherein the fetal monitor further comprises at least one of a sound module, a power switch, a power supply, a processor and a controller.

18. The method of claim 15 further comprising the step of placing the fetal monitor on an abdomen of an individual in a location adjacent a fetus to collect data after the step of providing the fetal monitor.

19. The method of claim 15, wherein the data collected includes at least one of a heartbeat from a fetus or a mother, a temperature of the mother, or a movement of the fetus.

20. The method of claim 15 further comprising the step of collecting data from the at least one other module after the step of controlling the fetal monitor.
